(19) United States
(12) Patent Application Publication
(10) Pub. No.: US 2013/0236508 A1
(43) Pub. Date: Sep. 12, 2013

(54) METHOD OF TREATMENT

(71) Applicants: Peter Hobman, Melbourne (AU); Kerrie Sanders, St Albans (AU); Michelle Rowney, Port Campbell (AU); Geoff Nicholson, Toowoomba (AU)

(72) Inventors: Peter Hobman, Melbourne (AU); Kerrie Sanders, St Albans (AU); Michelle Rowney, Port Campbell (AU); Geoff Nicholson, Toowoomba (AU)

(73) Assignee: MURRAY GOULBURN CO-OPERATIVE CO. LIMITED, Brunswick, Victoria (AU)

(21) Appl. No.: 13/806,266

(22) PCT Filed: Jun. 24, 2011
(86) PCT No.: PCT/AU11/00776

§ 371 Date: May 24, 2013

(30) Foreign Application Priority Data
Jun. 24, 2010 (AU) 2010902790

(51) Int. Cl. A61K 38/40; A23L 1/305; A23L 2/66; A61K 45/06

(52) U.S. Cl. CPC A61K 38/40; A61K 45/06; A23L 1/3056; A23L 2/66
USPC 424/400; 514/1.1

(57) ABSTRACT

The method relates to a method of treating proteinuria comprising administering lactoferrin.

### FIELD

The invention relates to methods of treating proteinuria or diseases associated with proteinuria.

### BACKGROUND

All references, including any patents or patent applications, cited in this specification are hereby incorporated by reference. It will be clearly understood that, although a number of prior art publications are referred to herein, this reference does not constitute an admission that any of these documents forms part of the common general knowledge in the art.

Urinary protein loss (proteinuria) affects some 100 million people worldwide and is a feature of kidney dysfunction, including inflammation, of glomerular origin. Proteinuria itself is a risk factor for both renal and extra-renal diseases.

Proteinuria usually reflects an increase in glomerular permeability for normally non-filtered plasma macromolecules such as albumin. Proteinuria occurs, in particular, in diseases of the renal tissue (nephritis, nephrosis, contracted kidney) and in engorged kidney as a result of cardiac insufficiency. In addition to the albumins (albuminuria), the globulins and other blood protein bodies also pass into the urine.

Kidney podocytes and their foot processes (FP) are a key component of the ultrafiltration system in the glomerulus where they comprise the filtration barrier together with endothelial cells and the glomerular basement membrane (GBM). Podocytes are located within the glomerulus of the kidney where they are attached to the GBM. Podocyte FPs are interconnected by the slit diaphragm (SD), a modified adherens junction. Proteinuric kidney diseases are typically associated with various degrees of podocyte membrane remodelling (FP effacement and/or SD disruption) driven by a rearrangement of the podocyte microfilament system.

Microalbuminuria, resulting from leakage of albumin across the glomerular podocyte filtration barrier into the urine, is considered a clinical window for a more generalised dysfunction in the systemic vasculature indicating a heightened risk of numerous diseases including metabolic syndrome, cardiovascular disease (CVD) or diabetes, for example.

Consequently, there exists a need for an improved and/or additional means for treating proteinuria or diseases associated with proteinuria.

### SUMMARY

A first aspect provides a method for treating proteinuria or diseases associated with proteinuria comprising administering to a subject in need thereof a therapeutically effective amount of lactoferrin.

A second aspect provides lactoferrin for treating proteinuria.

A third aspect provides use of lactoferrin in the manufacture of a medicament for treating proteinuria.

The method of the first aspect and use of the second and third aspects are based directly on the unexpected observation that lactoferrin is able to reduce proteinuria in subjects diagnosed with metabolic syndrome, particularly in a subgroup of subjects whose baseline urinary albumin/creatinine ratio (ACR) is greater than about 2 mg/mmol.

Any biologically active lactoferrin may be employed in the aspects of the invention. However, a fourth aspect provides orally ingestible or orally administrable lactoferrin for treating proteinuria.

The orally ingestible or orally administrable lactoferrin of the fourth aspect is suitable for administration according to the first aspect.

The current standard of care for treating proteinuria entails administration of angiotensin-converting enzyme (ACE) inhibitors or angiotensin receptor blockers (ARBs).

The present invention provides an alternative therapy for treating proteinuria. Alternatively, the present invention may delay the onset of the need for administering ACE inhibitors or ARBs. Alternatively, the present invention may be used in conjunction with ACE inhibitors or ARBs.

### BRIEF DESCRIPTION OF THE DRAWINGS

FIG. 1 depicts a schematic representation of the study design.

FIG. 2 plots the urinary albumin/creatinine ratio (ACR) in the subgroup of participants with urinary ACR>2 mg/mmol at baseline.

FIG. 3 plots the urinary ACR data from FIG. 2 for the lactoferrin group only.

FIG. 4 plots the percent change in urinary ACR data from FIG. 2 between visit 3 (V3) and visit 1 (V1).

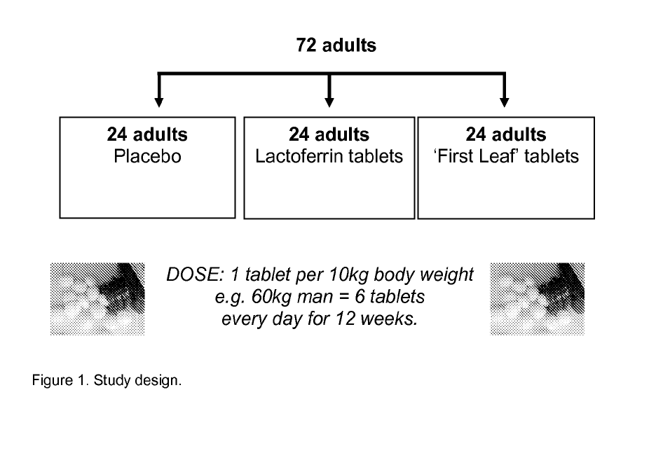

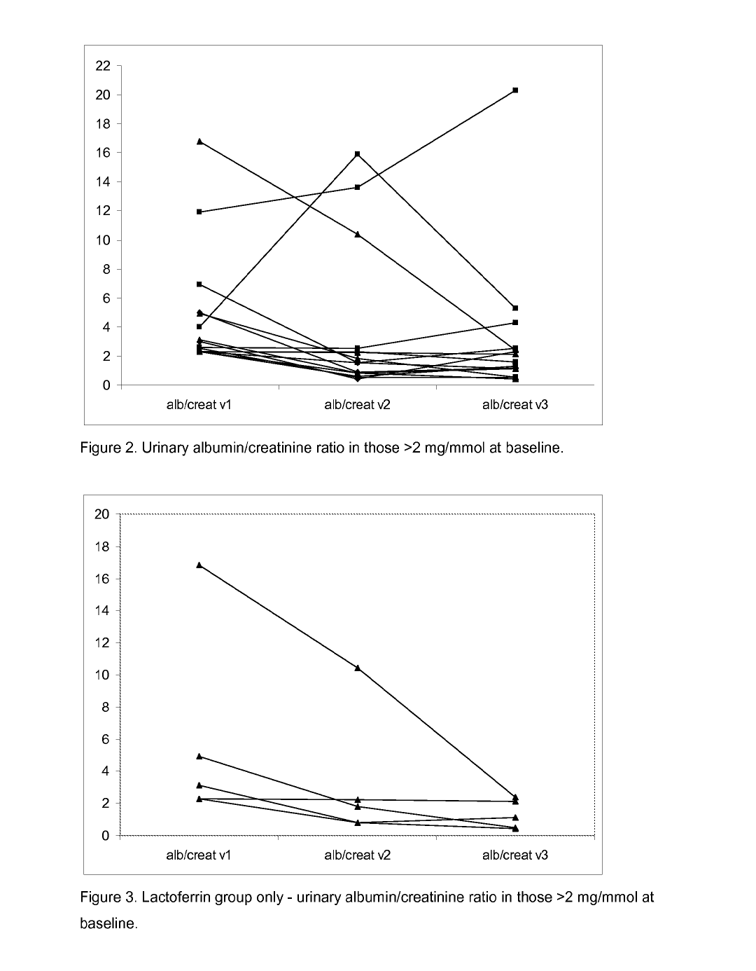

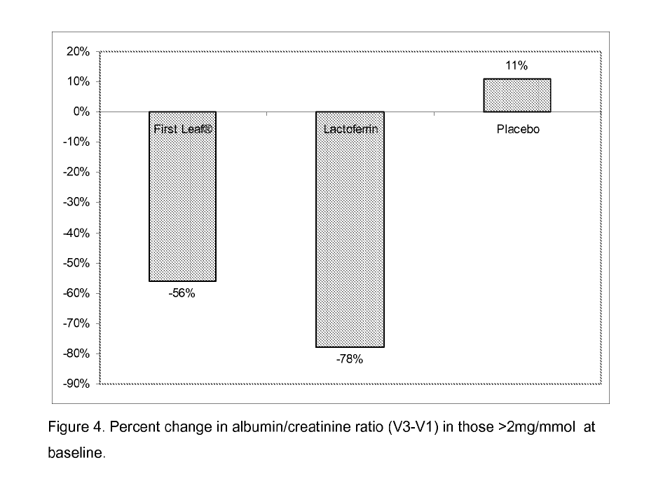

### DETAILED DESCRIPTION

The invention relates to the treatment of proteinuria using lactoferrin. The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage. Thus, for example, the present method of “treating” proteinuria of the first aspect encompasses both prevention of the condition or disorder in a predisposed individual and treatment of the condition or disorder in a clinically symptomatic individual. “Treating” as used herein covers any treatment of, or prevention of a condition or disorder in a vertebrate, a mammal, particularly a human, and includes: inhibiting the condition or disorder, i.e., arresting its development; or relieving or ameliorating the effects of the condition or disorder, i.e., cause regression of the effects of the condition or disorder.

As used herein, “symptom” refers to a phenomenon which arises from and accompanies a particular condition or disorder, i.e. underlying cause, and serves as an indication of that condition or disorder. A “symptom” may be directly observable in a subject, or may be indirectly observable, for example by use of a laboratory test or assay. Furthermore, as used herein, treatment of a symptom includes treatment of the underlying cause and treatment of the underlying cause includes treatment of the symptom.

“Prophylaxis” or “prophylactic” or “preventative” therapy as used herein includes preventing the condition from occurring or ameliorating the subsequent progression of the condition in a subject that may be predisposed to the condition, but has not yet been diagnosed as having it.

As used herein, the term “proteinuria” refers to the presence of protein in the urine in excess of normal levels. “Proteinuria” includes “albuminuria” and “microalbuminuria”. Normal human levels of protein appear in the urine in the range of about 0 to 30 mg/L, although for a random urine sample, the level may reach about 80 mg/L. For a 24 hour urine collection, normal human levels of urinary protein are in the range of about 0 to 150 mg. Proteinuria is indicated by a urinary ACR of greater than about 30 mg/mmol.

Proteinuria is “treated”, “ameliorated” or “reduced” when the level of proteinuria has dropped to at least about 10% below the baseline (for example, a value of 100 at baseline would drop at least about 10% to about 90). In some embodiments of the first to third aspects, the reduction of proteinuria is at least about 20%, 30%, 40%, 50%, 60%, 70%, 80%, 90%, 95%, 96%, 97%, 98%, 99% or more below baseline in the subject. In one embodiment of any one of the first to third aspects, the reduction of proteinuria is about 56% or about 78% below baseline in the subject. Reduction of proteinuria may be assessed about 1 week, or about 2, 3 or 4 weeks, or about 1 month, or about 2, 3, 4, 5, 6, 7, 8, 9, 10, 11 or 12 months, or about 1 year, or about 2 or 3 years or longer after initiation of treatment.

Proteinuria, including albuminuria and microalbuminuria, often leads to or is indicative of a disease, but is not limited to production of a disease. Proteinuria is intended to encompass all forms of proteinuria, including but not limited to physiological proteinuria; functional proteinuria; and athletic proteinuria, which relates to a form of functional proteinuria following excessive muscular exertion. Further, proteinuria covers benign proteinuria (also known as “essential” proteinuria), which refers to types or proteinuria that are not the result of pathologic changes in the kidneys. Proteinuria also covers pathologic proteinuria, for example levels of protein in the urine greater than normal physiological levels.

At the cellular level, proteinuria is accompanied by a structural rearrangement of podocyte cells. Renal ultrafiltration is located within the renal glomerulus, a combination of blood vessels and cells. Highly specialised podocyte cells perform the filtering work and are main target cells in kidney disease. Podocytes can reorganise their actin-based cytoskeleton in a highly dynamic fashion. Such a reorganisation determines the integrity of the ultrafiltration barrier in the kidney. Reorganisation of the actin cytoskeleton in podocyte foot processes from stress fibres into cortical actin leads to podocyte foot processes effacement and the development of proteinuria. Podocyte damage can be caused by many conditions and factors. These alterations lead to ongoing damage of the kidney and over time to a deterioration of the kidney function.

As used herein, the term “albuminuria” (also known as “macroalbuminuria”) refers to the presence of albumin in the urine in excess of normal levels. Since urinary protein is predominantly albumin, normal human levels of urinary ACR are in the range of about 0 to 30 mg/mmol.

As used herein, the term “microalbuminuria” refers to the presence of albumin in the urine, excreted at a rate of about 20 to 200 μg/min or at a level of about 30 to 300 mg/L in humans. When defined by the urinary ACR, “microalbuminuria” refers to a urinary ACR of greater than about 30 mg/g, or a urinary ACR of about 3.5 mg/mmol or greater for women and about 2.5 mg/mmol or greater for men. Microalbuminuria is often an early warning of kidney disease, but can also be present for other reasons.

“Creatinine” is a break-down product of creatine in muscle, and is usually produced at a fairly constant rate by the body (depending on muscle mass). Creatinine is chiefly filtered out of the blood by the kidneys (glomerular filtration and proximal tubular secretion). There is little-to-no tubular reabsorption of creatinine. Creatinine may be reported in units of mg/dL or μmol/L (1 mg/dL equals is 88.4 μmol/L). The typical human reference ranges for serum creatinine are 0.5 to 1.0 mg/dL (about 45 to 90 μmol/L) or 50 to 110 μmol/L for women and 0.7 to 1.2 mg/dL (60 to 110 μmol/L) or 60 to 120 μmol/L for men. Men tend to have higher levels of creatinine than women because men generally have more skeletal muscle mass than women.

If the filtering of the kidney is deficient, plasma creatinine levels rise. Therefore, creatinine levels in plasma and urine may be used to calculate the creatinine clearance (CrCl), which reflects the glomerular filtration rate (GFR). The GFR is clinically important because it is a measurement of renal function. However, in cases of severe renal dysfunction, the creatinine clearance rate will be overestimated because the active secretion of creatinine will account for a larger fraction of the total creatinine cleared. Ketoacids, cimetidine and trimethoprim reduce creatinine tubular secretion and therefore increase the accuracy of the GFR estimate, particularly in severe renal dysfunction.

A more complete estimation of renal function can be made by measuring the plasma creatinine concentration and the plasma urea concentration. Blood urea nitrogen (BUN)-to-creatinine ratio can indicate problems other than those intrinsic to the kidney; for example, a urea level raised out of proportion to the creatinine may indicate a pre-renal problem such as volume depletion. Plasma urea concentration may be expressed as BUN (to give BUN/creatinine ratio) or urea (to give urea/creatinine ratio). Normal human plasma values of BUN are 7 to 30 mg/dL or of urea 2.5 to 10.7 mmol/L.

As used herein, the “BUN/creatinine ratio” is the ratio (mg/g) of BUN (mg/dL) and plasma creatinine (mg/dL). Alternatively, the “urea/creatinine ratio” is the molar ratio of plasma urea (mmol/L) and plasma creatine (μmol/L)

The principle behind this ratio is the fact that both urea and creatinine are freely filtered by the glomerulus. However, urea reabsorption by the tubules can be regulated (increased or decreased), whereas creatinine reabsorption remains constant and minimal. Accordingly, a plasma BUN/creatinine ratio of greater than about 20 mg/mg or a urea/creatinine ratio of greater than about 100 mmol/mmol may be indicative of pre-renal disease (BUN reabsorption is increased and disproportionately elevated relative to creatinine in plasma). A plasma BUN/creatinine ratio of about 10 to 20 mg/mg or a urea/creatinine ratio of about 40 to 100 mmol/mmol may be indicative of normal or post-renal disease (BUN reabsorption is within normal limits). A plasma BUN/creatinine ratio of less than about 10 mg/mg or a urea/creatinine ratio of less than about 40 mmol/mmol may be indicative of intra-renal disease (renal damage causes decreased BUN reabsorption and BUN is disproportionately reduced relative to creatinine in plasma). However, an elevated BUN/creatinine ratio or urea/creatinine ratio due to low or low-normal creatinine and BUN or urea within the reference range is unlikely to be of clinical significance.

In an embodiment of any one of the first to third aspects, the proteinuria to be treated is associated with pre-renal disease, intra-renal disease (i.e. kidney disease), or post-kidney disease.

Methods to detect and diagnosis proteinuria, albuminuria or microalbuminuria are well known to one of skill in the art and include radioimmunoassays, immunoassays with latex bodies, fluoroimmunoassays, enzyme immunoassays, agglutination inhibition, immunoturbidimetry, immunonephelometry and radial immunodiffusion assays. Proteinuria, albuminuria or microalbuminuria can be measured by a special urine test either on a single urine sample or timed urine collection.

The person skilled in the art will be aware of enzyme-based and antibody-based methods for determining creatinine and urea concentrations.

In an embodiment of the first, second or third aspect, the proteinuria is associated with impaired glucose metabolism, impaired insulin action, metabolic syndrome, diabetes mellitus (diabetes), hypertension and/or obesity.

As used herein, “metabolic syndrome” refers to a collection of disorders that occur together and increase a subject's risk of developing type 2 diabetes, stroke or heart disease. The causes of metabolic syndrome are complex and not well understood, but there is thought to be a genetic link. Being overweight or obese and physically inactive increases risk of developing metabolic syndrome. Metabolic syndrome is also referred to as “syndrome X” or “insulin resistance syndrome”.

A subject is classed as having metabolic syndrome when they have:
Central (abdominal) obesity—excess fat in and around the stomach (abdomen)-plus any two of the following factors:
   hypertension;
   high blood triacylglycerol;
   low high density lipoproteins (HDL);
   impaired fasting glucose (blood glucose levels higher than normal but not high enough to be diagnosed as type 2 diabetes); and
   insulin resistance (the body does not use insulin as effectively as it should, especially in the muscles and liver).

As used herein, “insulin resistance” refers to the state in which the pancreas needs to release increasing levels of insulin to maintain normal blood glucose levels. Insulin resistance increases the risk of developing type 2 diabetes and is found in most people with this type of diabetes. If the pancreas is unable to produce extra insulin to overcome insulin resistance, blood glucose levels will rise and impaired glucose tolerance or diabetes will develop. Subjects with type 2 diabetes frequently also have other features of metabolic syndrome and a significantly increased risk of cardiovascular disease.

As used herein, “central obesity” is when the main deposits of body fat are around the abdomen and the upper body. Waist circumference is proportional to the risk developing metabolic syndrome. A subject's risk for central obesity varies depending on their gender and ethnic background. Generally, a waist circumference of 94 cm or more (men) or 80 cm or more (women) is indicative of risk for metabolic syndrome.

As used herein, “hypertension” refers to blood pressure greater than about 140/90 mmHg in human subjects in the absence of other risk factors. An ideal blood pressure range in human subjects is considered to be less than about 130/80 mmHg (or lower, if other diseases are present). Hypertension increases the risk of developing cardiovascular disease, stroke and kidney disease.

Triacylglycerol and cholesterol are lipids derived from the diet and are produced by the liver and are transported in the blood by lipoproteins. Increased blood triacylglycerol and decreased HDL cholesterol increase the risk for metabolic syndrome and atherosclerosis, which is a contributing factor in cardiovascular disease. Overweight or obesity is also a risk factor in itself for conditions such as high triacylglycerol levels, hypertension and atherosclerosis.

As used herein, “impaired glucose tolerance”, sometimes referred to as “pre-diabetes” refers to blood glucose levels that are higher than normal but not high enough to be called diabetes. One-third of subjects with untreated impaired glucose tolerance or impaired fasting glucose will develop diabetes.

Many of these conditions are interlinked in complicated ways and it is difficult to work out the chain of events. Some researchers consider that obesity could be the starting point for metabolic syndrome.

Diabetes mellitus defines a complex of metabolic diseases derived from multiple causative factors and is characterised by impaired glucose metabolism, usually associated with impaired protein and fat metabolism. This results in elevated fasting and postprandial serum glucose that leads to complications if left untreated. Four different forms of diabetes mellitus are known, (1) type 1 diabetes mellitus, (2) type 2 diabetes mellitus, (3) the so-called gestational diabetes mellitus, which begins or is recognised for the first time during pregnancy, and (4) some other forms which are mainly based on genetic defects.

The term “diabetes mellitus” includes, but is not limited to, metabolic abnormalities such as increased blood glucose level, obesity associated pathologies, impaired glucose tolerance, increased insulin resistance, hyperlipidemia, dyslipidemia, increase in cholesterol (hypercholesterinemia, hypertriglyceridemia), hyperinsulinemia, hypertension, and microalbuminuria. Impaired glucose tolerance and impaired fasting glucose are the two symptoms referred to as pre-diabetes. This stage is associated with the so-called insulin resistance, one of a group of metabolic diseases called “metabolic syndrome” or “syndrome X”, particularly associated with a high fat to muscle ratio. Since type 2 diabetes mellitus is often associated with other symptoms from syndrome X, such as hypertriglyceridemia or dyslipidemia.

The two major forms of diabetes mellitus are the type 1 and type 2 diabetes mellitus, of which type 2 diabetes mellitus is the most prevailing form. Type 1 and type 2 diabetes mellitus are associated with hyperglycemia, hypercholesterolemia and hyperlipidemia. The insensitivity to insulin and absolute insulin deficiency in type 1 and 2 diabetes mellitus leads to a decrease in glucose utilisation by the liver, muscle and the adipose tissue and to increased blood glucose levels. Uncontrolled hyperglycemia is associated with the dysfunction and failure of various organs such as the eyes, heart, blood vessels, kidney and nerves thus leading to increased and premature mortality due to an increased risk for microvascular and macrovascular diseases, including nephropathy, neuropathy, retinopathy, ulceration of the legs and feet, fatty liver disease, hypertension, cardiovascular diseases, and cerebrovascular diseases (stroke), the so-called diabetic complications.

Type 1 diabetes mellitus is the form of diabetes mellitus which usually begins with childhood or puberty and is characterised by an auto-immune destruction of the insulin-producing β-cells leading to a complete deficiency of insulin secretion.

Type 2 diabetes mellitus is the form of diabetes mellitus which occurs predominantly in adults in whom adequate production of insulin is available in the early stage of the diseases, yet a defect exists in insulin sensitivity, especially in insulin-mediated utilisation and metabolism of glucose in peripheral tissues. The changes in various tissues associated with type 2 diabetes mellitus exist even before clinical symptoms are detected.

Also contemplated is the treatment of insulin resistance induced by trauma (e.g. burns or nitrogen imbalance) and adipose tissue disorders (e.g. obesity).

Furthermore, the proteinuria to be treated according to any one of the first to third aspects includes, but is not limited to, proteinuria associated with (i.e. arise from or give rise to): acute nephritic syndrome; Alport's syndrome; aminoaciduria; amyloidosis; benign orthostatic (postural) proteinuria; bladder tumour; cardiovascular disease; collagen vascular diseases (e.g. systemic lupus erythematosus); congestive heart failure; dehydration; diabetes; diabetic nephropathy; ebola hemorrhagic fever; eclampsia; Fabry's disease; Fanconi syndrome; Focal segmental glomerulosclerosis (FSGS); Glomerular diseases (e.g. membranous glomerulonephritis); glomerulonephritis; Goodpasture syndrome; heavy metal ingestion; hemoglobinuria; hemolytic-uremic syndrome (HUS); HIV-associated nephropathy; hypertension; hypertensive nephrosclerosis; IgA nephropathy (i.e. Berger's disease); IgM nephropathy; infections (e.g. systemic infection, HIV, syphilis, hepatitis, post-streptococcal infection); interstitial nephritis; kidney (renal) disease; kidney (renal) distress; lupus erythematosus; malignant hypertension; medullary cystic kidney disease; membranoproliferative glomerulonephritis I and II; membranous nephropathy; mesangioproliferative glomerulonephritis; minimal change disease; multiple myeloma; myoglobinuria; Nail Patella syndrome; necrotising vasculitis; nephritic syndromes; nephrotic syndromes (i.e. intrinsic renal failure); nephrotoxic drugs; organ rejection; pancreatic distress; polycystic kidney disease; post-infectious glomerulonephritis (e.g. post-streptococcal glomerulonephritis); preeclampsia; rapidly progressive (crescentic) glomerulonephritis; reflux nephropathy; renal vein thrombosis; sarcoidosis; sickle cell disease; strenuous exercise; stress; toxic lesions of kidneys; urinary distress; urinary tract infection; and vascular endothelial dysfunction.

With severe proteinuria, general hypoproteinemia can develop and it results in diminished oncotic pressure (ascites, edema, hydrothorax).

Each year in the United States, more than 100,000 people are diagnosed with kidney failure. Kidney failure (or end stage renal disease, ESRD) is the final stage of chronic kidney disease (CKD). Diabetes, hypertension, and nephritis or renal inflammation are the most common causes of kidney disease in the United States and Australia (34%, 14%, and 22% of new patients in Australia, respectively). CKD and proteinuria are recognised to be amongst the most significant clinical risk factors for CVD, hospitalisation, and all-cause mortality.

Kidney disease or damage that results as a complication of diabetes is called diabetic nephropathy, which is characterised by glomerular hyperfiltration, extracellular matrix accumulation, glomerular enlargement, mesangial expansion, and intertubular fibrosis, resulting ultimately in diabetic glomerulosclerosis and progressive renal failure. Early diagnosis of diabetes and early intervention are critical in slowing the progression towards renal failure seen in many type 1 and a significant percentage of type 2 diabetic subjects. Kidney failure originated from diabetes accounts for nearly 44% of new cases of kidney failure in the United States and 34% of new cases of CKD in Australia. Nearly 24 million people in the United States have diabetes and 7.6% of the population, or approximately 1.5 million people, in Australia have diabetes. Even when diabetes is controlled, the disease can lead to CKD and kidney failure. In the United States, around 20 to 30 percent of people with diabetes develop diabetic nephropathy, and nearly 180,000 people eventually end up with kidney failure.

Renal dysfunction may contribute to overall CVD by promoting vascular thickening and vascular calcification, as well as by activating inflammatory pathways. It is also recognised that insulin resistance is closely associated with oxidant stress, early decline in renal function and albuminuria. Recently, proteinuria and microalbuminuria have been identified as risk factors for CVD.

Hypertension is another common cause for kidney disease. People with diabetes are prone to hypertension. There are many pharmaceutical research efforts aiming to aggressively slow down the progression of chronic kidney disease.

CKD has two important abnormal clinical test results: decreased glomerular filtration rate (GFR, estimated GFR less than about 60 mL/min) and proteinuria. For example, levels of urinary albumin excretion greater than normal are observed frequently in patients with type 2 diabetes. Diabetic kidney disease takes many years to develop. Over several years, people who are developing kidney disease will have small amounts of the blood protein albumin begin to leak into their urine. Moderately increased levels of albuminuria, so-called microalbuminuria, are predictive both of progressive renal function loss up to diabetic nephropathy, and of cardiovascular morbidity and mortality. As the disease progresses, more protein, including albumin, leaks into the urine. This stage is termed as macroalbuminuria or proteinuria. As the amount of protein in the urine increases, the kidneys' filtering function usually begins to drop. As kidney damage develops, blood pressure often rises as well.

The lactoferrin to be used according to any one of the first to third aspects, or the lactoferrin of the fourth aspect, includes, but is not limited to, lactoferrin, mutant lactoferrins, truncated lactoferrins, lactoferrin lobes or fusions of any of the above to other peptides, polypeptides, proteins or hydrolysates of lactoferrin.

Lactoferrin is an 80 kD iron-binding glycoprotein present in most exocrine fluids, including tears, bile, bronchial mucus, gastrointestinal fluids, cervico-vaginal mucus, seminal fluid, and milk. It is a major constituent of the secondary specific granules of circulating poly-morphonuclear neutrophils. The richest source of lactoferrin is mammalian milk and colostrum.

Lactoferrin circulates at a concentration of 2 to 7 μg/mL. It has multiple postulated biological roles, including regulation of iron metabolism, immune function, and embryonic development. Lactoferrin has anti-microbial activity against a range of pathogens including Gram positive and Gram negative bacteria and fungi, including yeasts. The anti-microbial effect of lactoferrin is based on its capability of binding iron, which is essential for the growth of the pathogens. Lactoferrin also inhibits the replication of several viruses and increases the susceptibility of some bacteria to antibiotics and lysozyme by binding to lipid A component of lipopolysaccharides on bacterial membranes.

As used herein, “lactoferrin” refers to pure lactoferrin, naturally derived, recombinant or synthetic lactoferrin, fragments of lactoferrin, variants of lactoferrin, hydrolysates of lactoferrin, or any mixture or combination thereof.

The lactoferrin may be a pure lactoferrin polypeptide containing no more than two (i.e., 0, 1, or 2) metal ions per molecule.

The lactoferrin may be isolated or purified. “Isolated” or “purified” lactoferrin is substantially free of at least one agent or compound with which it is naturally associated. For instance, an isolated protein is substantially free of at least some cellular material or contaminating protein from the cell or tissue source from which it is derived. The phrase “substantially free of cellular material” refers to preparations where the lactoferrin is at least 50 to 59% (w/w) pure, at least 60 to 69% (w/w) pure, at least 70 to 79% (w/w) pure, at least 80-89% (w/w) pure, at least 90 to 95% pure, or at least 96%, 97%, 98%, 99% or 100% (w/w) pure.

The purity of a polypeptide can be measured by any appropriate standard method, for example, by column chromatography, polyacrylamide gel electrophoresis, or HPLC analysis. Practically, the measurement of the ion/lactoferrin ratio for a preparation of lactoferrin can be in the range of 0-2.5.

The lactoferrin can be a naturally occurring polypeptide, e.g. isolated from milk, a recombinant polypeptide, or a synthetic polypeptide. Recombinant lactoferrin may be produced by expression in cell-free expression systems or in transgenic animals, plants, fungi or bacteria, or other useful species. Recombinant human lactoferrin is available from ProSpec Protein Specialists. Alternatively, lactoferrin may be produced using known organic synthetic methods. The lactoferrin may be isolated from milk by cation exchange chromatography followed by ultrafiltration and diafiltration.

Useful lactoferrin fragments include individual components of hydrolysates of lactoferrin, fragments that include either or both the N and C lobe, fragments of the N- or G-lobes, lactoferricin and fragments generated (by artificial or natural processes) and identified by known techniques as discussed below.

The lactoferrin can be of a mammalian origin.

Verified sequences of bovine and human lactotransferrins (lactoferrin precursors), lactoferrins and peptides therein can be found in Swiss-Prot (http://au.expasy.org/cgi-bin/sprot-search-ful).

The lactoferrin may include, for example bovine lactotransferrin precursor accession number P24627 or its fragment bovine Lactoferricin B, or human lactotransferrin precursor accession number P02788 or its fragments Kaliocin-1, Lactoferroxin A, Lactoferroxin B, or Lactoferroxin. Other examples of lactoferrin amino acid and mRNA sequences that have been reported and are useful in any one of the first to fourth aspects include, but are not limited to: the amino acid (Accession Numbers AAW71443 and NP_{—}002334) and mRNA (Accession Number NM_{—}002343) sequences of human lactoferrin; the amino acid (Accession Numbers NP_{—}851341 and CAA38572) and mRNA (Accession Numbers X54801 and NM_{—}180998) sequences of bovine lactoferrin; the amino acid (Accession Numbers JC2323, CAA55517 and AAA97958) and mRNA (Accession Number U53857) sequences of goat lactoferrin; the amino acid (Accession Number CAA09407) and mRNA (Accession Number AJ010930) sequences of horse lactoferrin; the amino acid (Accession Number NP_{—}001020033) and mRNA (Accession Number NM_{—}001024862) sequences of sheep lactoferrin; the amino acid (Accession Numbers NP_{—}999527, AAL40161 and AAP70487) and mRNA (Accession Number NM_{—}214362) sequences of pig lactoferrin; the amino acid (Accession Numbers NP_{—}032548 and A28438) and mRNA (Accession Number NM_{—}008522) sequences of mouse lactoferrin; the amino acid (Accession Number CAA06441) and mRNA (Accession Number AJ005203) sequences of water buffalo lactoferrin; and the amino acid (Accession Number CAB53387) and mRNA (Accession Number AJ131674) sequences of camel lactoferrin. These sequences may be used in wild type or variant form.

Accordingly, in an embodiment of any one of the first to fourth aspects the lactoferrin is sheep, goat, pig, mouse, water buffalo, camel, yak, horse, donkey, llama, bovine or human lactoferrin. In another embodiment, the lactoferrin is buffalo or deer lactoferrin. An animal from which lactoferrin may be produced may be a transgenic animal designed to over-express lactoferrin in its milk.

Variants of a wild-type lactoferrin polypeptide (e.g., a fragment of the wild-type lactoferrin polypeptide containing at least 2 (e.g., 4, 6, 8, 10, 20, 50, 100, 200, 300, 400, 500, 600, 700) amino acids, or a recombinant protein containing a lactoferrin polypeptide sequence) that maintain the biological activity of a wild-type lactoferrin polypeptide may be employed. Alternatively, the lactoferrin can be produced using genetic engineering or chemical synthesis techniques well-known in the art.

As used herein, the term “variant” refers to a naturally occurring (an allelic variant, for example) or non-naturally occurring (an artificially generated mutant, for example) lactoferrin that varies from the predominant wild-type amino acid sequence of a lactoferrin of a given species by the addition, deletion or substitution of one or more amino acids. Methods for generating such variants are known in the art. Useful recombinant lactoferrins and lactoferrin fragments and methods of producing them are reported in U.S. patent specifications U.S. Pat. No. 5,571,691, U.S. Pat. No. 5,571,697, U.S. Pat. No. 5,571,896, U.S. Pat. No. 5,766,939, U.S. Pat. No. 5,849,881, U.S. Pat. No. 5,849,885, U.S. Pat. No. 5,861,491, U.S. Pat. No. 5,919,913, U.S. Pat. No. 5,955,316, U.S. Pat. No. 6,066,469, U.S. Pat. No. 6,080,599, U.S. Pat. No. 6,100,054, U.S. Pat. No. 6,111,081, U.S. Pat. No. 6,228,614, U.S. Pat. No. 6,277,817, U.S. Pat. No. 6,333,311, U.S. Pat. No. 6,455,687, U.S. Pat. No. 6,569,831, U.S. Pat. No. 6,635,447, US 2005-0064546 and US 2005-0114911. Useful variants also include bovine lactoferrin variants bLf-a and bLf-b.

It is understood by one of ordinary skill in the art that certain amino acids may be substituted for other amino acids in a protein structure without adversely affecting the activity of lactoferrin. It is thus contemplated by the inventors that various changes may be made in the amino acid sequences of lactoferrin without appreciable loss of their biological utility or activity. Such changes may include deletions, insertions, truncations, substitutions, fusions, shuffling of motif sequences, and the like.

One of skill in the art will recognise that lactoferrin may contain any number of conservative changes its amino acid sequence without altering its biological properties to produce a “variant”. Such conservative amino acid modifications are based on the relative similarity of the amino acid side-chain substituents, for example, their hydrophobicity, hydrophilicity, charge, size, and the like. Exemplary conservative substitutions which take various of the foregoing characteristics into consideration are well known to those of skill in the art and include arginine and lysine; glutamate and aspartate; serine and threonine; glutamine and asparagine; and valine, leucine, and isoleucine.

Also included within the meaning of the term “variant” are homologues of lactoferrin. A homologue is typically a polypeptide from a different species but sharing substantially the same biological function or activity as the corresponding polypeptide. Variants may share at least 50%, 55%, 60%, 65%, 70%, 75%, 80%, 85%, 90%, 95%, 96%, 97%, 98% or 99% sequence identity.

Persons skilled in the art would readily appreciate the numerous software packages to enable them to design or identify homologues of the lactoferrin nucleotide and amino acid sequences, for example the “BLAST” program or other suitable packages.

Variant lactoferrin may be generated by techniques including but not limited to techniques for mutating wild type proteins such as, but not limited to: site-directed mutagenesis of a wild-type nucleotide sequence encoding lactoferrin and expression of the resulting polynucleotide; techniques for generating expressible polynucleotide fragments such as PCR using a pool of random or selected primers; techniques for full or partial proteolysis or hydrolysis of wild type or variant lactoferrin polypeptides; and techniques for chemical synthesis of polypeptides. Variants or fragments of lactoferrin may be prepared by expression as recombinant molecules from lactoferrin DNA or RNA, or variants or fragments thereof. Nucleic acid sequences encoding variants or fragments of lactoferrin may be inserted into a suitable vector for expression in a cell, including eukaryotic cells such as, but not limited to, Aspergillus or bacterial cells such as but not limited to E. coli. Lactoferrin variants or fragments may be prepared using known PCR techniques including but not limited to error-prone PCR and DNA shuffling. Error-prone PCR is a process for performing PCR under conditions where the copying fidelity of the DNA polymerase is low, such that a high rate of point mutations is obtained along the entire length of the PCR product. DNA shuffling refers to forced homologous recombination between DNA molecules of different but highly related DNA sequence in vitro, caused by random fragmentation of the DNA molecule based on sequence homology, followed by fixation of the crossover by primer extension in a PCR reaction. Variants or fragments of lactoferrin may also be generated by known organic synthetic methods.

The lactoferrin used in the first to fourth aspects can contain an iron ion (as in a naturally occurring lactoferrin polypeptide) or a non-iron metal ion (e.g., a copper ion, a chromium ion, a cobalt ion, a manganese ion, a zinc ion, or a magnesium ion). For instance, lactoferrin isolated from bovine milk can be depleted of iron and then loaded with another type of metal ion. For example, copper loading can be achieved according to the same method for iron loading described above. Methods for loading lactoferrin with other metal ions are known in the art.

A preparation of lactoferrin (e.g., lactoferrin isolated from bovine milk) can contain polypeptides of a single species, e.g., every molecule binding two iron ions. It can also contain polypeptides of different species, e.g., some molecules binding no ion and others each binding one or two ions; some molecules each binding an iron ion and others each binding a copper ion; some molecules each being a biological active lactoferrin polypeptide (full-length or shorter than full-length) that contains 0, 1, or 2 metal ions and others each being a fragment (same or different) of the polypeptide; or all molecules each being a fragment (same or different) of a full-length lactoferrin polypeptide that contains 0, 1, or 2 metal ions.

Metal ion-binding fragments of lactoferrin may be obtained by known techniques for isolating metal-binding polypeptides including, but not limited to, metal affinity chromatography. Fragments of lactoferrin may be contacted with free or immobilised metal ions, such as Fe³⁺ and purified in a suitable fashion. For example, fragments may be contacted at neutral pH with a metal ion immobilised by chelation to a chromatography matrix comprising iminodiacetic acid or tris(carboxymethyl)-ethylenediamine ligands. Bound fragments may be eluted from the supporting matrix and collected by reducing the pH and ionic strength of the buffer employed. Metal-bound fragments may be prepared according to methods known in the art.

A mixture of full-length lactoferrin polypeptides and various fragments of full-length lactoferrin polypeptides can be prepared from a hydrolysate, e.g., a partial digest such as a proteinase digest, of full-length lactoferrin polypeptides. A mixture of various fragments of full-length lactoferrin polypeptides, can be prepared, for example, by complete digestion (i.e., no full-length polypeptides remain after digestion) of full-length lactoferrin polypeptides, or by mixing different fragments of full-length lactoferrin polypeptides. The degree of digestion can be controlled according to methods well known in the art, e.g., by manipulating the amount of proteinase or the time of incubation. Otherwise, a mixture of full-length lactoferrin polypeptides and various fragments of full-length lactoferrin polypeptides can be obtained by mixing full-length lactoferrin polypeptides with various fragments of full-length lactoferrin polypeptides (e.g., synthetic fragments).

In one embodiment of any one the first to fourth aspects, the lactoferrin comprises a full or partial enzyme hydrolysate (including but not limited to a protease, trypsin, chymotrypsin, chymosin, plasmin, pepsin, papain, peptidase, or aminopeptidase hydrolysates), a full or partial microorganism hydrolysate (including but not limited to hydrolysis by a bacterium from the genera Bacillus, Bifidus, Enterococcus, Lactobacillus, Lactococcus, Leuconostoc, Pediococcus, Propionbacter, Pseudomonas or Streptococcus or a mixture thereof), a full or partial acid hydrolysate (including but not limited to trifluoro acetate and hydrochloric acid hydrolysates), a cyanogen bromide hydrolysate, or a mixture thereof.

The lactoferrin hydrolysate may be a hydrolysate of a natural, recombinant or synthetic lactoferrin polypeptide or a mixture thereof. The lactoferrin hydrolysate may be a human lactoferrin hydrolysate or a bovine lactoferrin hydrolysate or mixtures thereof.

The lactoferrin may be non-glycosylated or glycosylated. The lactoferrin may be fully or partially glycosylated with naturally occurring or non-naturally occurring glycosyl groups. In addition the lactoferrin may be modified, for example by conjugation to a polymer to increase its circulating half-life such as by pegylation or other chemical modification. It may also be desirable to introduce a modification to lactoferrin to improve storage stability. Such modified lactoferrin is also envisaged for use according to the first to fourth aspects.

The lactoferrin may comprise about 50 to 100% by weight, or at least about 50, 55, 60, 65, 70, 75, 80, 85, 90, 95 or 99% by weight, of lactoferrin.

The following is an exemplary procedure for isolating lactoferrin from bovine milk.

Fresh skim milk (7 L, pH 6.5) is passed through a 300 ml column of S Sepharose Fast Flow equilibrated in milli Q water, at a flow rate of 5 ml/min and at 4° C. Unbound protein is washed through with 2.5 bed volumes of water and bound protein eluted stepwise with approximately 2.5 bed volumes each of 0.1 M, 0.35 M, and 1.0 M sodium chloride. Lactoferrin eluting as a discreet pink band in 1 M sodium chloride is collected as a single fraction and dialysed against milli Q water followed by freeze-drying. The freeze-dried powder is dissolved in 25 mM sodium phosphate buffer, pH 6.5 and subjected to rechromatography on S Sepharose Fast Flow with a sodium chloride gradient to 1 M in the above buffer and at a flow rate of 3 ml/min. Fractions containing lactoferrin of sufficient purity as determined by gel electrophoresis and reversed phase HPLC are combined, dialysed and freeze-dried. Final purification of lactoferrin is accomplished by gel filtration on Sephacryl 300 in 80 mM dipotassium phosphate, pH 8.6, containing 0.15 M potassium chloride. Selected fractions are combined, dialysed against milli Q water, and freeze-dried. The purity of this preparation is greater than 95% as indicated by HPLC analysis and by the spectral ratio values (280 nm/465 nm) of ^{˜}19 or less for the iron-saturated form of lactoferrin.

Iron saturation is achieved by addition of a 2:1 molar excess of 5 mM ferric nitrilotriacetate to a 1% solution of the purified lactoferrin in 50 mM Tris, pH 7.8 containing 10 mM sodium bicarbonate. Excess ferric nitrilotriacetate is removed by dialysis against 100 volumes of milli Q water (twice renewed) for a total of 20 hours at 4° C. The iron-loaded (holo-) lactoferrin is then freeze-dried.

Iron-depleted (apo-) lactoferrin is prepared by dialysis of a 1% solution of the highly purified lactoferrin sample in water against 30 volumes of 0.1 M citric acid, pH 2.3, containing 500 mg/L disodium EDTA, for 30 h at 4° C. Citrate and EDTA are then removed by dialysis against 30 volumes of milli Q water (once renewed) and the resulting colourless solution freeze-dried.

In an embodiment of any one of the first to third aspects, the lactoferrin is orally ingestible or orally administrable.

The orally ingestible or orally administrable lactoferrin may be encapsulated, microencapsulated or nanoencapsulated.

The lactoferrin used in any one of the aspects may be provided as a nutracuetical or a pharmaceutical.

The term “nutraceutical” as used herein refers to an edible product that may be isolated or purified from food, e.g. a milk product, which is demonstrated to have a physiological benefit or to provide protection or attenuation of an acute or chronic disease or injury when orally administered. The nutraceutical may thus be presented in the form of a dietary preparation or supplement, either alone or admixed with edible foods or drinks.

“Nutracueticals” are also referred to as “functional foods”. Nutraceuticals can be produced by various methods and processes known in the art including, but not limited to, synthesis (chemical or microbial), extraction from a biological material, mixing functional ingredient or component to a regular food product, fermentation or using a biotechnological process. A nutraceutical may exert its effects directly in the body or it may function e.g. through intestinal bacterial flora.

Generally, although not entirely necessary, such nutraceuticals will contain lactoferrin purified to some degree, or at the very least, all components of the nutraceutical, for example “First Leaf”, will be verifiable.

The nutraceutical is an example of the medicament manufactured according to the third aspect or the lactoferrin of the fourth aspect.

Examples of suitable foods, drinks or edible consumer products include soluble powders, milk powders, confectionary, reconstituted fruit products, breakfast cereals, ready-to-eat bars, snack bars, muesli bars, spreads, dips, diary products including yoghurts and cheeses, a liquid or a ready-to-drink formulation including dairy and non-dairy based drinks (e.g. milks, juices, teas, or soft drinks), food supplements, a dietary supplements (e.g., a hard or soft capsule, a mini-bag, or a tablet, a tea-bag), nutritional formulations, sports nutrition supplements including dairy and non-dairy based sports nutrition supplements, an infant formula, particularly a humanised milk formula for administration to infants, food additives such as protein sprinkles and dietary supplement products including daily supplement tablets.

The nutraceutical preferably has acceptable sensory properties (such as acceptable smell, taste and palatability).

The nutraceutical may be produced as is conventional; for example, the composition may be prepared by blending together the protein and other additives, for example, various flavours, fibres, sweeteners, and other additives may also be present. If used, an emulsifier may be included in the blend. The nutraceutical may include other nutrients such as amino acids, a protein, or a carbohydrate. Additional vitamins and minerals may be added at this point but are usually added later to avoid thermal degradation. Further vitamins and/or minerals may be selected from at least one of vitamins A, B1, B2, B3, B5, B6, B11, B12, biotin, C, D, E, H and K and calcium, magnesium, potassium, zinc and iron.

If it is desired to produce a powdered nutraceutical, the lactoferrin may be admixed with additional components in powdered form. The powder should have a moisture content of less than about 5% by weight. Water, preferably water which has been subjected to reverse osmosis, may then be mixed in to form a liquid mixture.

If the nutraceutical is to be provided in a ready to consume liquid form, it may be heated in order to reduce the bacterial load. If it is desired to produce a liquid nutraceutical composition, the liquid mixture is preferably aseptically filled into suitable containers. Aseptic filling of the containers may be carried out using techniques commonly available in the art. Suitable apparatus for carrying out aseptic filling of this nature is commercially available.

Preferably the nutraceutical also comprises one or more pharmaceutically acceptable carriers, diluents or excipients. Nutraceuticals may comprise buffers such as neutral buffered saline, phosphate buffered saline and the like; carbohydrates such as glucose, mannose, sucrose, lactose, lactulose, or dextrans; mannitol or lactitol; proteins; polypeptides or amino acids such as glycine; antioxidants; chelating agents such as EDTA; adjuvants and preservatives.

As bovine milk is a natural product that has been in food chain for hundreds of years, the lactoferrin used as a nutraceutical need not be totally pure. However, to reduce the amount of composition to be administered it is preferred that the lactoferrin is concentrated significantly with respect to its concentration in milk. Preferably the lactoferrin is administered in at a concentration of at least 10 times its concentration in milk and more preferably 20, 30, 40, or 50 times its concentration in milk.

A pharmaceutical formulation is one which is suitable for administration to humans. A veterinary formulation is one that is suitable for administration to animals. Generally such formulations will contain purified lactoferrin or at the very least all components of the formulation, for example “First Leaf”, will be verifiable.

The formulations are examples of the medicament manufactured according to the third aspect or the lactoferrin of the fourth aspect.

The medicament manufactured according to the third aspect or the lactoferrin of the fourth aspect may comprise one or more carriers and optionally other therapeutic agents. Each carrier, diluent, adjuvant and/or excipient may be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “pharmaceutically acceptable” salt or ester of a novel compound as provided herein is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

The formulations may be administered orally, topically, or parenterally in formulations containing conventional non-toxic pharmaceutically acceptable carriers, adjuvants, and vehicles. Preferably, the formulations are administered orally.

The formulations may be administered orally as tablets, aqueous or oily suspensions, lozenges, troches, powders, granules, emulsions, capsules, syrups or elixirs. The formulation for oral use may contain one or more agents selected from the group of sweetening agents, flavouring agents, colouring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

In one example, tablets can be formulated in accordance with conventional procedures by compressing mixtures of the lactoferrin with a solid carrier and a lubricant. Examples of solid carriers include starch and sugar bentonite. The lactoferrin can also be administered in the form of a hard shell tablet or a capsule containing a binder, e.g., lactose or mannitol, a conventional filler, and a tableting agent.

The term parenteral as used herein includes intravenous, intraarterial, intraperitoneal, intramuscular, subcutaneous, subconjunctival, intracavity, transdermal and subcutaneous injection, aerosol for administration to lungs or nasal cavity or administration by infusion by, for example, osmotic pump.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The formulations may also contain other active compounds providing supplemental, additional, or enhanced therapeutic functions. The formulations may also be included in a container, pack, or dispenser together with instructions for administration.

The formulations may be presented for use in the form of veterinary formulations, which may be prepared, for example, by methods that are conventional in the art. Examples of such veterinary formulations include those adapted for:
(a) oral administration, external application, for example drenches (e.g. aqueous or non-aqueous solutions or suspensions); tablets or boluses; powders, granules or pellets for admixture with feed stuffs; pastes for application to the tongue, particularly adapted for protection through the rumen if to be administered to ruminants;
(b) parenteral administration for example by subcutaneous, intramuscular or intravenous injection, e.g. as a sterile solution or suspension; or (when appropriate) by intramammary injection where a suspension or solution is introduced in the udder via the teat;
(c) topical applications, e.g. as a cream, ointment or spray applied to the skin; or
(d) intravaginally, e.g. as a pessary, cream or foam.

It is especially advantageous to formulate the formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The formulations are to be administered in therapeutically effective amounts. As used herein, an “effective amount” of lactoferrin is a dosage which is sufficient to reduce proteinuria.

Generally, a therapeutical effective amount may vary with the subject's age, condition, and sex, as well as the severity of the medical condition in the subject. The dosage may be determined by a physician and adjusted, as necessary, to suit observed effects of the treatment. Appropriate dosages for administering lactoferrin may range from 5 mg to 100 mg, from 15 mg to 85 mg, from 30 mg to 70 mg, or from 40 mg to 60 mg, 5 mg to 500 mg, 10 mg to 400 mg, 20 mg to 300 mg, 25 mg to 250 mg, 40 mg to 200 mg, 50 mg to 100 mg. For example, doses may be 5, 10, 15, 20, 25, 30, 35, 40, 45, 50, 60, 70, 80, 90, 100, 120, 125, 150, 175, 200, 225, 230, or 250 mg. The formulations can be administered in one dose, or at intervals such as once daily, twice daily, once weekly, and once monthly.

Dosage schedules can be adjusted depending on the half life of lactoferrin, or the severity of proteinuria.

Generally, the formulations are administered as a bolus dose, to maximise the circulating levels of lactoferrin for the greatest length of time after the dose. Continuous infusion may also be used after the bolus dose.

There is growing evidence that proteinuria is not only the result (a disease marker) of kidney damage and/or disease progression but also is a direct cause of or risk factor for kidney damage and/or progression of renal disease. There are evidences to indicate that renal injury from proteinuria occurs through multiple pathways, including induction of tubular chemokine expression and complement activation that lead to inflammatory cell infiltration in the interstitium and sustained fibrogenesis. Macrophages are prominent in the interstitial inflammatory infiltrate. This cell type mediates progression of renal injury to the extent that macrophage numbers in renal biopsy predict renal survival in patients with chronic renal disease. Chemoattractants and adhesive molecules for inflammatory cells are up-regulated by excess ultrafiltered protein load of proximal tubular cells. Therefore, therapies to reduce proteinuria are a common clinical practice for slowing the progressing of kidney diseases.

Currently, the drugs useful for reducing proteinuria are drugs used for treating hypertension. Two types of drugs, ACE inhibitors and ARBs, have proven effective in reducing proteinuria levels and slowing the progression of kidney disease. Although the exact pharmacological mechanisms of ACE/ARB in reducing proteinuria are not clear, no data indicate they are involved in inflammatory processes.

Accordingly, an embodiment of the first or second aspect further comprises administering an ACE inhibitor or an ARB, or administering lactoferrin to a subject being treated with an ACE inhibitor or an ARB. In an embodiment of the third aspect, the medicament is formulated for use with an ACE inhibitor or an ARB.

If ingested or administered as separate formulations, lactoferrin may be used to treat proteinuria before and/or concurrently and/or following treatment with an ACE inhibitor or ARB.

Other therapeutically useful agents beneficial for proteinuria or a related condition or disease may optionally be included in or administered simultaneously or sequentially with the lactoferrin.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### EXAMPLE

Summary of Clinical Trial

The ‘First Leaf’ Lactoferrin Study was a randomised clinical trial involving three treatment groups: First Leaf; Lactoferrin and placebo. The placebo group acted as the comparator and the trial was a randomised double-blind design. The allocation of treatment group was decided independently of the research team and both the participants and researchers did not know which group were taking which treatment until the 12-week intervention period was completed. Each participant was assigned a unique study number. A statistician randomised participants into the three treatment groups by using their unique study number in a statistical software program ‘Minitab’™. The clinical trial pharmacist dispensed the medication based on these three groups.

‘First Leaf’ is a health supplement developed by FOURLEAF JAPAN Co. Ltd. In addition to other ingredients each tablet contains 25 mg lactoferrin. This lactoferrin is supplied by MG Nutritionals, a subsidiary of Murray Goulburn Co-op Ltd. The project was conducted according to the protocol and to the standards of the International Clinical Harmonisation Guidelines for Clinical Trials.

Ethical Issues and Tolerance of Study Medication

No unanticipated issues emerged in the course of the project, including serious adverse events, unexpected adverse incidents, or effects on participants.

However, one female participant ceased study medication due to extensive body rash that developed soon after commencement of study medication. The rash gradually resolved after cessation of the medication. Four other participants temporarily stopped taking study medication (duration 4 to 18 days) due to gastrointestinal symptoms. In all four cases, symptoms resolved and did not reoccur following resumption of the medication.

No complaints were received concerning the conduct of the research.

Aims

The study was a randomised double-blind clinical trial investigating the effects of ‘First Leaf’ and lactoferrin on health parameters indicative of risk of chronic diseases commonly related to lifestyle and the environment. Over a 12-week intervention period men and women with metabolic syndrome were given one of three treatments (matched capsules):—(1) ‘First Leaf’; (2) Lactoferrin only or (3) Placebo. Changes in clinical and biochemical markers over the intervention period are compared between the three groups.

Introduction

‘First Leaf’

‘First Leaf’ is a supplement commonly taken by Japanese adults as a health supplement for an enhanced feeling of wellbeing and improved immunity. It contains extracts of Marigold, cassis and an herbal tea, Eyebright. Ingredients include lycopene, lactoferrin, coenzyme Q₁₀ and lutein. Lycopene and lutein are carotenoids thought to provide health benefits through their role as antioxidants. Coenzyme Q₁₀ also known as ubiquinone, is a vitamin-like substance particularly associated with protection against heart disease.

Lactoferrin

Lactoferrin is an iron-binding glycoprotein with potent antimicrobial and immunomodulatory activities. Recently lactoferrin has also been shown to promote bone growth by stimulating the proliferation and differentiation of osteoblasts and inhibiting osteoclastogenesis. Consequently it is now thought that lactoferrin may have a physiological role in bone growth and healing, and a potential therapeutic role as an anabolic factor in osteoporosis. Although lactoferrin is extracted from the whey milk, it is also a naturally occurring glycoprotein found in small concentrations in the secondary granules of neutrophils. Animal studies have shown that systemic levels of lactoferrin dramatically increase during inflammation and it is thought that lactoferrin may play a role in counter-balancing the catabolic effects on the skeleton from some of the mediators of the inflammatory response. It is suggested that the mechanism of action for lactoferrin also contains a component for differential regulation of cellular immune responses. Activation of this mechanism is likely to change the proportion of different T helper cells with the ratio of helper T1 to helper T2 cells (Th1/Th2) providing an index of allergy symptoms.

Study Objectives

Primary Efficacy Objective

The primary objective of the study was to compare the change in clinical and biochemical markers related to metabolic syndrome, osteoporosis and immunological functioning between the ‘First Leaf’-treated group (active), tactoferrin'-treated group (active) and the placebo (control) group during intervention of 12-weeks.

Subjects and Methods

The study was conducted in accordance with the National Statement on Ethical Conduct in Research Involving Humans (Commonwealth of Australia 2001), the ICH Good Clinical Practice guidelines, and the Declaration of Helsinki (1996). The study was approved by the Barwon Health Research and Ethics Advisory Committee (06/09) and all participants signed the study informed consent form.

Inclusion Criteria

All participants fulfilled the Australian criteria for metabolic syndrome and were overweight. Most had a body mass index in the obese category (>30). Specifically, all subjects met the following criteria to be eligible for the trial:
Ambulatory, outpatient males and females aged 25 years and over;
Body mass index (BMI) 25 to 35 kg/m² (BMI=weight [kg] divided by height [metres] squared);
At screening, all participants had Metabolic Syndrome:
   All have central obesity defined as waist circumference 94 cm for Europid men and 80 cm for Europid women, with ethnicity specific values for other groups;
   plus any two of the following four factors:
1. Raised serum triglyceride level 1.7 mmol/L);
2. Reduced serum HDL cholesterol level (<1.03 mmol/L in males and <1.29 mmol/L in females);
3. Raised blood pressure (BP) (systolic BP 130 mmHg or diastolic BP 85 mmHg or treatment of previously diagnosed hypertension); and
4. Impaired fasting glycaemia (fasting plasma glucose 5.6 mmol/L and 7.0 mmol/L without a previous diagnosis of type 2 diabetes).

Exclusion Criteria
Known allergy to milk protein;
BMI<25 OR>35 kg/m²;
Currently taking, or have taken for more than one month in the past 6 months, any of the following medication: oral hypoglycaemic agents; insulin; bisphosphonates; selective estrogen receptor modulators; strontium; parathyroid hormone (PTH); calcitriol or estrogen (excluding vaginal estrogen cream);
Glucocorticoid therapy equivalent to prednisolone>5 mg daily for more than six-months during the preceding three years;
Previous diagnosis of the following medical conditions: diabetes; rheumatoid arthritis; Pagets disease; multiple myeloma; cancer with the exception of non-invasive non-melanoma skin cancer and cervical carcinoma-in-situ; significant renal disease or plasma creatinine>150 μmol/L; hypercalcaemia (corrected plasma calcium>2.65 mmol/L); sarcoidosis; hyperthyroidism (in the last 2 years); or
Active peptic ulcer disease or inflammatory bowel disease.

Dose

The dose of study medication for all participants was 1 capsule per 10 kg body weight (weight at visit one). Since the average weight of participants was 89 kg, an average dose was nine capsules per day. Participants were advised to take half the dose in the morning and half in the evenings.

The placebo was lactose powder with no active ingredient. Each ‘First Leaf’ tablet contains 25.5 mg lactoferrin plus other active ingredients. One standard ‘First Leaf’ tablet was equivalent to one capsule. Each lactoferrin capsule contained 125 mg lactoferrin, i.e. approximately 5-fold more lactoferrin per capsule than each ‘First Leaf’ capsule.

Assessments

Participants attended the Clinical Trials Unit at Geelong hospital for three study visits—Visit 1=baseline; Visit 2=six weeks and 3=twelve weeks after commencement of study medication. Pathology and clinical assessments including anthropometry were performed at each study visit. Pathology samples were centrifuged and aliquoted as appropriate, prior to immediate storage at −80° C.

Participants were asked about allergic symptoms that have experienced in the past 6 weeks and also in the past 12 months.

The study medication was prepared and dispensed by the clinical trials pharmacist at Geelong Hospital at visits one and two. Participants were given six weeks supply plus an extra one week's supply in case their next study visit needed to be rescheduled. All unused study medication was returned and counted at the next study visit. Compliance was calculated as a percentage of capsules taken compared with the recommended dose of capsules.

Statistical Analysis

The primary endpoint was the change in clinical and biochemical markers from baseline to three months between the three groups. The analysis tested if the mean of the dependent variable for either the ‘First Leaf’ or Lactoferrin groups was different from the placebo group. This was potentially meaningful if the mean values were not different at baseline (visit 1) but were different from the placebo group at visit 2 and/or visit 3. Treatment effects were evaluated using an analysis of variance with significance defined at P<0.05. Any difference in means subsequent to baseline (visit 1) was further investigated by looking at the ‘change in the dependent variable since baseline’ (i.e. visit 3-visit 1).

Analysis of variance compares population means for more than two groups. A two-way analysis of variance simultaneously tests for differences in the mean between treatment groups and between the baseline visit and the study visit at 6 and 12 weeks intervention. The data was analysed by two-way analysis of variance using a general linear model for unbalanced data. This method simultaneously tests for differences in mean values in the following combinations:
(A) Treatment groups: Gp 1 vs. Gp 2; Gp 1 vs Gp 3 and Gp 2 vs. Gp 3
(B) Visit No.: V1 vs. V2; V1 vs. V3; V2 vs. V3

Parameters not normally distributed were transformed (log natural etc) with tests for equal variance done in addition to testing for interaction between the treatment groups and the visit number.

The null hypothesis tested:

H₀: μ₁=μ₂=μ₃: The mean value of each parameter (dependent variable) is equal in groups 1, 2 and 3.

H₁: The ‘dependent variable’ means are not equal between groups 1, 2 and 3. Post-hoc analyses were then performed as appropriate.

Ad hoc analysis with Tukey's adjustment for multiple testing was performed if p values were less than 0.05. This second procedure identified if the difference between the three treatment groups occurred after intervention (i.e. visit 2 and/or 3).

The questionnaire was structured to be categorical. Questions asked at baseline were analysed as the proportion of participants in each treatment group indicating an improved status compared with their answer at visit one. All responses were analysed by the Chi-square test of proportions i.e. was the proportion of participants who improved different between the three treatment groups?.

The statistical analyses were performed using a statistical software program (Minitab™, version 13).

A timeline of the study is presented in Table 1.

TABLE 1
Timeline

| | |
|---|---|
| Commencement date | 21/04/06 |
| Total number of participants consented | 77 |
| Date first participant entered | 16/05/06 |
| Date last participant entered | 27/07/06 |
| Date first participant commenced study medication | 27/07/06 |
| Date last participant commenced study medication | 03/08/06 |
| Date first participant completed study (visit 3) | 05/10/06 |
| Date last participant completed study (visit 3) | 03/11/06 |

Results

For ease of interpretation, the tables do not include visit 2 (V2) results (week six) unless the trend differed from the visit three (week twelve) result. The analysis of variance included the week six results to test for differences between the three treatment groups.

Participants

Participant numbers and characteristics are presented in Tables 2 and 3, respectively.

TABLE 2
Participant Numbers

| Number | Female | Male | Total | Withdrawals |
|---|---|---|---|---|
| Screened | 208 | 61 | 269 | |
| Eligible | | | 107 | |
| Ineligible | | | 161 | |
| Withdrew prior to consent | | | 31 | |
| Consented | 59 | 18 | 77 | 1 |
| Visit 1 completed | 59 | 17 | 76 | 4 |
| Visit 2 completed | 54 | 15 | 69* | 1 |
| Visit 3 completed | 57 | 14 | 71 | |

*2 people unable to obtain adequate blood sample at visit 2.

TABLE 3
Participant Characteristics by Gender

| | | | Visit Placebo | Lactoferrin | First Leaf | Normal range |
|---|---|---|---|---|---|---|
| | | | mean (+SD) OR median [range] | | | |
| Gender | Male | 1 | 3 | 6 | 5 | |
| | Female | 1 | 21 | 18 | 18 | |
| Weight (kg) | Male | 1 | 99.3 (1.2) | 95.1 (10.8) | 97.8 (11.9) | |
| | Female | 1 | 88.5 (11.3) | 86.3 (13.8) | 86.9 (10.3) | |
| BMI | Male | 1 | 32.8 (2.8) | 30.1 (2.9) | 32.0 (9.1) | |
| | Female | 1 | 33.1 (3.3) | 32.2 (3.0) | 32.4 (3.0) | 20-25 |
| Age (years) | Male | 1 | 56.6 (7.5) | 54.2 (13.0) | 59.5 (9.1) | |
| | Female | 1 | 55.9 (11.7) | 59.0 (10.2) | 53.4 (10.4) | |
| Compliance¹ | | | 95.8% | 87.5% | 90.3% | |

¹Compliance refers at least 80% of tablets were taken.

Liver Function and Protein Metabolism

Serum levels of total protein, bilirubin and the liver enzymes (Table 4) gamma glutamyl transpeptidase (GGT) and alanine transaminase (ALT) did not differ between the three treatment groups (p=0.9 to 0.3) and did not change over the study period (p=0.5 to 0.8). Aspartate transaminase (AST) levels did not differ at baseline but increased in both placebo and ‘First Leaf’ groups during the study (p=0.05). The mean increase in both groups was 2 U/L by 12 weeks. Serum albumin was higher in the lactoferrin group at baseline (41 vs. 42 vs. 41; placebo; lactoferrin and First Leaf, respectively. p=0.01) and decreased in all three groups by week 12 (39 vs. 40 vs. 40; placebo; lactoferrin and First Leaf, respectively. p=0.01). Serum levels of the enzyme alkaline phosphatase differed at baseline (p=0.02) but levels did not change over the 12 weeks (p=0.44).

TABLE 4
Liver Function and Protein Metabolism

| | | Visit | Placebo | Lactoferrin | First Leaf | Normal | By treatment | By visit | |
|---|---|---|---|---|---|---|---|---|---|
| Analyte | Unit | mean (+SD) OR median [range] | | | | | P value | | |
| Total protein | g/L | 1 | 74 (4) | 73 (4) | 72 (4) | 63-82 | 0.91 | 0.46 | |
| | | 3 | 73 (5) | 72 (4) | 73 (4) | | | | |
| Albumin | g/L | 1 | 41 (3) | 42 (3) | 41 (2) | 34-50 | 0.01 | <0.001 | All 3 groups decreased over 12 wk |
| | | 3 | 39 (3) | 40 (3) | 40 (2) | | | | |
| Alkaline phosphatase | U/L | 1 | 82 (31) | 87 (21) | 78 (16) | 20-120 | 0.02 | 0.44 | Difference between groups at |
| | | 3 | 83 (27) | 88 (21) | 78 (16) | | | | baseline |
| GGT | U/L | 1 | 30 [2-172] | 21 [8-61] | 19 [8-197] | <30 | 0.26 | 0.82 | log nat rank |
| | | 3 | 29 [2-113] | 22 [6-74] | 18 [8-159] | | | | |
| AST | U/L | 1 | 22 [15-43] | 25 [14-70] | 24 [15-49] | <35 | 0.86 | 0.05 | log nat |
| | | 3 | 25 [17-52] | 25 [15-82] | 26 [17-68] | | | | |
| ALT | U/L | 1 | 32 [20-57] | 32 [17-153] | 34 [15-79] | <40 | 0.94 | 0.76 | log nat |
| | | 3 | 29 [18-71] | 30 [13-233] | 30 [15-118] | | | | |
| Bilirubin | μmol/L | 1 | 10 (6) | 11 (4) | 11 (4) | 1-20 | 0.55 | 0.55 | |
| | | 3 | 10 (6) | 11 (4) | 10 (4) | | | | |

Lipids

High serum cholesterol is a major risk factor for cardiovascular disease. The Australian Heart Foundation recommends that total cholesterol is less than 5.5 mmol/L (http://www.nevdgp.org.au/info/heartf/docs/hhd3.htm). The total serum cholesterol is largely a combination of low-density lipoprotein (LDL) and high-density lipoprotein (HDL) cholesterol. It is the LDL cholesterol that is associated with ‘clogging’ blood vessels and it is recommended that this level is less than 3.5 mmol/L. The mean level of LDL cholesterol in study participants was on the upper limit of the ‘normal’ range and did not differ throughout the intervention (Table 5). The HDL cholesterol can help to ‘unclog’ the arteries. The combination of high HDL and low LDL cholesterol is desirable. The recommended level of HDL cholesterol is more than 1 mmol/L. The baseline level of HDL cholesterol in the participants was lower in the placebo group. This is associated with fewer males since males tend to have lower HDL cholesterol than females. HDL cholesterol did not change in any of the three treatment groups throughout the study. This also means that the placebo group had a lower cholesterol/HDL ratio than the other two groups although there were no differences in the change in ratio throughout the study. Triacylglycerol (TAG) is a type of fat occurring in blood. It is formed from the digestion of fats and alcohol and it is recommended that the TAG level is less than 2 mmol/L. Throughout the 12-week study, the TAG levels decreased in all three groups. This is not clinically meaningful since it also occurred in those taking the placebo.

Electrolytes and Renal Function

The change in serum levels of the following electrolytes and indicators of renal function (Table 6) did not differ between the three treatment groups over the 12 week study: sodium (p=0.56); potassium (p=0.41); chloride (p=0.51); bicarbonate (p=0.41); urea (p=0.85); serum creatinine (p=0.5), urinary albumin (p=0.99) and urinary creatinine (p=0.3). The urinary ACR was a non-parametric distribution. The median albumin/creatinine ratio decreased in both the lactoferrin and ‘First Leaf’ groups between baseline and 12-weeks (40%, p=0.39 and 50%, p=0.16, lactoferrin and First Leaf, respectively compared with 20% placebo group).

TABLE 5
Lipids

| | | Visit | Placebo | Lactoferrin | First Leaf | Normal | By treatment | By visit | |
|---|---|---|---|---|---|---|---|---|---|
| Analyte | Unit | mean (+SD) | | | | | P value | | |
| Cholesterol | mmol/L | 1 | 5.4 (1.1) | 5.5 (0.8) | 5.3 (0.9) | <5.5 | 0.61 | 0.28 | |
| | | 3 | 5.0 (1.0) | 5.2 (0.8) | 5.3 (0.9) | | | | |
| Triglyceride | mmol/L | 1 | 1.8 (0.6) | 1.9 (0.6) | 1.8 (0.8) | 0.3-2.0 | 0.14 | 0.012 | All 3 groups decreased over time |
| | | 3 | 1.4 (0.5) | 1.5 (0.5) | 1.6 (0.9) | | | | |
| HDL cholesterol | mmol/L | 1 | 1.3 (0.3) | 1.1 (0.2) | 1.2 (0.3) | 1.0-3.0 | 0.007 | 0.99 | Different between groups at baseline |
| | | 3 | 1.3 (0.3) | 1.1 (0.2) | 1.3 (0.3) | | | | |
| LDL cholesterol | mmol/L | 1 | 3.3 (1.0) | 3.5 (0.6) | 3.3 (0.6) | <4.0 | 0.33 | 0.7 | |
| | | 3 | 3.1 (0.9) | 3.3 (0.7) | 3.3 (0.7) | | | | |
| Cholesterol/HDL | | 1 | 4.2 (0.9) | 4.9 (0.7) | 4.5 (1.2) | 1.0-5.0 | 0.000 | 0.28 | Lactoferrin different from placebo |
| cholesterol ratio | | 3 | 4.0 (0.9) | 4.6 (0.8) | 4.3 (0.9) | | | | (p = 0.0002) Trend (p = 0.24) lower in v3 |

TABLE 6
Electrolytes and Renal Function

| | | Visit | Placebo | Lactoferrin | First Leaf | Normal | By treatment | |
|---|---|---|---|---|---|---|---|---|
| Analyte | Unit | mean (+SD) OR median [range] | | | | | P value | |
| sodium | mmol/L | 1 | 139 (2) | 139 (2) | 139 (2) | 136-148 | | |
| | | 3 | 137 (2) | 138 (2) | 138 (3) | | 0.56 | One-way GLM on change |
| potassium | mmol/L | 1 | 4.1 (0.3) | 4.1 (0.4) | 4.1 (0.2) | 3.5-5.0 | | between v3-v1 |
| | | 3 | 4.1 (0.3) | 4.2 (0.4) | 4.1 (0.2) | | 0.41 | One-way GLM on change |
| chloride | mmol/L | 1 | 102 (2) | 103 (2) | 103 (2) | 98-107 | | between v3-v1 |
| | | 3 | 101 (3) | 101.8 (1.7) | 102 (2) | | 0.51 | One-way GLM on change |
| bicarbonate | mmol/L | 1 | 28.1 (2.4) | 27.8 (3.5) | 27.4 (2.7) | 22-32 | | between v3-v1 |
| | | 3 | 26.9 (2) | 27.4 (2.4) | 26.0 (2.3) | | 0.41 | One-way GLM on change |
| urea | mmol/L | 1 | 5.7 (1.6) | 5.8 (1.4) | 5.8 (1.7) | 2.5-7.5 | | between v3-v1 |
| | | 3 | 5.7 (1.6) | 5.7 (1.5) | 5.8 (1.6) | | 0.85 | One-way GLM on change |
| serum creatinine | μmol/L | 1 | 69.5 (18.0) | 71.3 (13.3) | 73.7 (19) | 30-120 | | between v3-v1 |
| | | 3 | 64.3 (14.6) | 68.1 (12.8) | 71.5 (21.0) | | 0.5 | One-way GLM on change |
| urinary creatinine | mmol/L | 1 | 12.6 (9.4) | 11.3 (7.4) | 15.6 (8.1) | 7.0-25.0 | | between v3-v1 |
| (Cr) | | 3 | 9.3 (9.0) | 11.2 (6.5) | 10.2 (5.4) | | 0.3 | One-way ANOVA on change |
| urinary albumin | mg/L | 1 | 10.0 [2-121] | 7.0 [2-155] | 10.0 [2-107] | 0-20 | | between v3-v1 |
| (Alb) | | 3 | 6 [2-77] | 7.5 [2-96] | 6.0 [2-36] | | 0.99 | One-way GLM on change |
| urinary Alb/Cr ratio | mg/mmol | 1 | 1.1 [0.3-11.9] | 1.0 [0.3-16.8] | 1.2 [0.3-5.0] | 0.0-3.4 | | between v3-v1 |
| | | 2 | 0.7 [0.3-15.9] | 0.5 [0.3-10.4] | 0.7 [0.2-2.6] | | | |
| | | 3 | 0.9 [0.3-20.3] | 0.6 [0.3-15.7] | 0.6 [0.3-2.6] | | 0.594 | One-way GLM on |
| | | | | | | | | change between v3-v1 |
| | | | | | | [p = 0.25] | 0.39 | Mann-Whitney placebo vs |
| | | | | | | | | lactoferrin v3-v1 |
| | | | | | | | 0.16 | Mann-Whitney placebo vs |
| | | | | | | | | ‘First Leaf’ v3-v1 |

Further analyses of participants with a high urinary albumin/creatinine ratio at baseline (over 2 mg/mmol; Table 7) suggests the greatest reductions in the ratio occurred in those with higher baseline values in the lactoferrin and ‘First Leaf’ groups but no reduction in the ratio in the placebo group (p=0.19).

TABLE 7
Urinary albumin/creatinine ratio (ACR) in subjects greater
than 2 mg/mmol ACR at baseline.

| | Placebo | Lactoferrin | First Leaf |
|---|---|---|---|
| n | 7 | 5 | 3 |
| Baseline: V1 | 4.6 (±3.6) | 5.9 (±6.2) | 3.4 (±1.4) |
| 12-Week: V3 | 5.1 (±6.9) | 1.3 (±0.9) | 1.5 (±0.7) |

Iron Studies

The placebo group with more females had lower baseline levels of the following parameters: haemaglobulin; iron; transferrin and percent transferrin saturation (p=0.007 to 0.01; Table 8). All three treatment groups decreased in iron levels over the 12-week study (p=0.06). The decrease was not different from placebo in either the lactoferrin or ‘First Leaf’ groups (p=0.66).

TABLE 8
Iron Studies

| | | Visit | Placebo | Lactoferrin | First Leaf | Normal | By treatment | By visit | |
|---|---|---|---|---|---|---|---|---|---|
| Analyte | Unit | mean (+SD) OR median [range] | | | | | P value | | |
| Hb | g/L | 1 | 139 (9) | 144 (11) | 143 (11) | 115-170 | 0.007 | 0.76 | |
| | | 3 | 138 (10) | 144 (11) | 140 (11) | | | | |
| Iron | μmol/L | 1 | 15.7 (3.6) | 18.1 (4.0) | 16.2 (5.3) | 7-30 | 0.017 | 0.06 | p = 0.04 baseline difference between |
| | | 3 | 14.8 (4.1) | 15.6 (4.1) | 14.9 (4.6) | | | | placebo and lactoferrin groups; |
| | | | | | | | | | Change in iron by treatment |
| | | | | | | | | | p = 0.662 |
| Ferritin | μg/L | 1 | 115 [23-266] | 101 [21-569] | 82 [7-691] | 10-200 | 0.42 | 0.91 | square root |
| | | 3 | 99 [21-249] | 101 [21-556] | 80 [7-909] | | | | |
| Transferrin | g/L | 1 | 2.7 (0.4) | 2.6 (0.3) | 2.7 (0.4) | 2.0-3.6 | 0.011 | 0.68 | p = 0.01 baseline difference between |
| | | 3 | 2.7 (0.4) | 2.5 (0.3) | 2.7 (0.5) | | | | lactoferrin group and other 2 groups |
| | | | | | | | | | Change in transferrin by treatment |
| | | | | | | | | | p = 0.7 |
| Transferrin | % | 1 | 27.6 (5.6) | 33.5 (8.4) | 29.1 (11.6) | 16-55 | 0.01 | 0.68 | |
| saturation | | 3 | 26.5 (8.1) | 29.8 (7.8) | 27.0 (10.1) | | | | |

Discussion

Participants

Fewer males responded to the recruitment advertisements for the study (61 compared with 208 females). Eighteen fulfilled the study criteria and signed the informed consent. The dropout rate was higher in males than females (22% compared with 5%). This resulted in the placebo group having a lower proportion of males compared with the other two groups upon completion of the three study visits (12.5% vs. 25% vs. 22%; placebo; lactoferrin; First Leaf, respectively). Those that withdrew tended to be younger (median and range {years}: 39.5 {27 to 62} vs. 56.5 {30.7 to 79.5} withdrew vs. participants who completed study).

Liver Function and Protein Metabolism

Fatty liver, with or without raised serum levels of hepatic enzymes, is common in obesity and has been associated with insulin resistance and the metabolic syndrome.

Electrolytes and Renal Function

There was no difference in serum electrolyte levels between the three groups. It was not expected that the intervention would be associated with changes in electrolytes but the results support the safety of this dose of oral lactoferrin and the ‘First Leaf’ supplement.

The urinary albumin to creatinine ratio is a useful measure of renal function used in diabetic renal disease. Relatively small incremental changes in this ratio have been shown to be strongly predictive of cardiovascular events. Microalbuminuria is indicated where there is an albumin: creatinine ratio greater than 2.5 mg/mmol in males or 3.5 mg/mmol in females. Proteinuria is indicated by a ratio of greater than 30 mg/mmol and is indicative of serious renal dysfunction.

Although the median urinary albumin creatinine ratio decreased slightly over the 12 weeks in the placebo group (18% or 0.2 mg/mmol) the median value decreased by 40 and 50 percent in the lactoferrin and ‘First Leaf’ groups. The greatest reductions occurred in those with higher baseline values (greater than 2 mg/mmol). When the analysis was restricted to these participants (n=15) there was no reduction in the placebo group compared with a decrease of 75 and 57 percent in the lactoferrin and ‘First Leaf’ groups. Confirmation of this result in a larger study using participants with high baseline urinary albumin to creatinine ratios would have clinical significance and could lead to therapeutic benefits.

Iron Studies

Lactoferrin is an iron-binding glycoprotein that has potent antimicrobial and immunodulatory activities. The protein belongs to the transferrin family and acts as an iron-chelator. It is present in adult serum in microscopic amounts. Functional lactoferrin receptors have now been identified in certain cell types (including osteoblasts) but it is not confirmed whether lactoferrin as a pharmaceutical or ‘neutraceutical’ agent has a therapeutic effect on the iron status metabolism in adults. Our results did not demonstrate significant changes in parameters of iron status. These results differ between males and females but the study was not powered to analysis the data by gender. The results from the placebo group compared with the lactoferrin and ‘First Leaf’ groups need to be interpreted with caution due to the lower ratio of males.

### CONCLUDING COMMENT

The results of this 12-week intervention placebo-controlled study have provided evidence that the supplements are safe at this dose level and have also highlighted areas of clinical and therapeutic interest, specifically, the decrease in the urinary albumin/creatinine ratio in both the ‘First Leaf’ and lactoferrin groups but in particular, the improved response in those on lactoferrin with a higher baseline albumin/creatinine ratio. Further studies with a longer duration of intervention and with specific targeting of recruitment to participants with certain baseline characteristics are recommended and likely to produce clinically meaningful findings. The study results suggest the target areas of recruitment should be people who have microalbuminuria.

1. A method of treating proteinuria comprising administering to a subject in need thereof a therapeutically effective amount of lactoferrin.

2. The method of claim 1, wherein the proteinuria is albuminuria or microalbuminuria.

3. The method of claim 1, wherein the proteinuria is diagnosed using a urinary albumin/creatinine ratio (ACR).

4. The method of claim 3, wherein the ACR of the subject when the proteinuria is diagnosed is greater than or equal to about 2 mg/mmol.

5. The method of claim 1, wherein the proteinuria is diagnosed using a blood urea nitrogen (BUN)/creatinine ratio or a urea/creatinine ratio.

6. The method of claim 1, further comprising administering to the subject an ACE inhibitor or an ARB.

7. The method of claim 1, wherein the lactoferrin is administered orally.

8. The method of claim 7, wherein the lactoferrin is administered orally as a food, a drink, a supplement, a nutraceutical, or a medicament.

9. The method of claim 7, wherein the lactoferrin that is administered orally is encapsulated, microencapsulated or nanoencapsulated.
